(19) United States
(12) Patent Application Publication
(10) Pub. No.: US 2011/0300586 A1
(43) Pub. Date: Dec. 8, 2011

(54) Two-Stage Process for Biomass Pretreatment

(76) Inventors: Chaogang Liu, Hanover, NH (US); Kevin Wenger, Hanover, NH (US)

(21) Appl. No.: 13/133,539

(22) PCT Filed: Dec. 18, 2009
(86) PCT No.: PCT/US2009/068738

§ 371 Date: Jul. 27, 2011

Related U.S. Application Data
(60) Provisional application No. 61/139,059, filed on Dec. 19, 2008.

(51) Int. Cl. C12P 19/14; D21C 3/06; D21C 3/04

(52) U.S. Cl. 435/99; 162/76; 162/80; 162/83

(57) ABSTRACT

Described herein are improved methods of pretreating lignocellulosic biomass. One aspect of the invention relates to a two-stage pretreatment process comprising a relatively low severity steam treatment, a controlled pH pretreatment, or autohydrolysis, followed by hydrolysis with dilute acid or hot water at a relatively low temperature. In certain embodiments, the methods increase hemicellulose sugar yields, substrate digestibility, and suitability for fermentation as compared to steam explosion or acid hydrolysis alone. The two-stage pretreatment processes also employ few chemicals, minimizing the costs associated with pretreatment of lignocellulosic biomass. Moreover, the two-stage pretreatment process may expand the range of suitable feedstocks for bioethanol production.

### RELATED APPLICATIONS

This application claims the benefit of priority to U.S. Provisional Patent Application Ser. No. 61/139,059, filed Dec. 19, 2008; the contents of which are hereby incorporated by reference.

### BACKGROUND OF THE INVENTION

The production of ethanol from biomass typically involves the breakdown or hydrolysis of lignocellulose-containing materials, such as wood, into disaccharides, such as cellobiose, and ultimately monosaccharides, such as glucose and xylose. Microbial agents, including yeasts, then convert the monosaccharides into ethanol in a fermentation reaction which can occur over a period of several days or weeks. Thermal, chemical and/or mechanical pretreatment of the lignocellulose-containing materials can shorten the required fermentation time and improve the yield of ethanol. Since the advent of the first alkaline pretreatment processes in the early 1900s, based on impregnation with sodium hydroxide, which improved the digestibility of straw, many pretreatment processes have been developed for lignocellulosic materials.

Hydrothermal pretreatment processes are among the most commonly used for improving the accessibility of these materials to enzymes. An example of such a hydrothermal process is described in Shell International Research's Spanish patent ES87/6829, which uses steam at a temperature of 200-250° C. in a hermetically sealed reactor to treat previously ground biomass. In this process, the reactor is cooled gradually to ambient temperature once the biomass is treated. Hydrothermal treatment that includes a sudden depressurization of the reactor, called steam explosion treatment, is one of the most effective pretreatment techniques when it comes to reducing particle size and solubilizing a fraction of the hemicellulose and lignin, thereby facilitating the eventual action of cellulolytic enzymes.

However, a significant fraction of hemicellulose sugars (in some cases more than 25%) may be damaged by the harsh conditions of steam explosion pretreatment. Moreover, sugar degradation products produced during steam explosion, such as furfural, HMF, and lignin are inhibitory to the microorganisms and enzymes used in subsequent processing steps (e.g., enzymatic hydrolysis and fermentation). Further, some studies have shown that steam explosion pretreatment is not effective for softwoods (Clark and Mackie, J. Wood Chem. & Tech., 1987, 7:373-403; Saddler et al., 1991). As an alternative, hydrolysis with dilute acids has been investigated due the associated relatively inexpensive chemical costs, high hemicellulose sugar yields (e.g., ˜90%), and effectiveness for pretreatment of almost all lignocellulosic biomass (e.g., woody and herbaceous feedstock). However, pretreatment process based solely on treatment with dilute acids can be economically prohibitive, due to the fact that they require relatively high capital and disposal costs.

It is therefore an object of this invention to provide biomass pretreatment processes that combine the best features of steam explosion and dilute acid hydrolysis, while minimizing their limitations. Other objects of the invention will be apparent from the following disclosure, claims, and drawings.

### SUMMARY OF THE INVENTION

In certain embodiments, this invention relates to an improved method of pretreating lignocellulosic biomass. In some embodiments the invention relates to a two-stage pretreatment process. In certain embodiments, the two-stage pretreatment process may comprise a relatively low severity steam treatment or autohydrolysis, followed by hydrolysis with dilute acid or hot water at a relatively low temperature. In other embodiments, the two-stage pretreatment process may comprise a controlled pH pretreatment or autohydrolysis, followed by hydrolysis with dilute acid or hot water at a relatively low temperature. In some embodiments, the methods can increase hemicellulose sugar yields, substrate digestibility, and fermentability in comparison to steam explosion or acid hydrolysis alone. The two-stage pretreatment process may also use fewer chemicals, lowering the cost associated with the pretreatment of lignocellulosic biomass. The two-stage pretreatment process may also reduce the overall energy costs associated with pretreatment of biomass. Moreover, the two-stage pretreatment process may expand the range of suitable feedstocks for bioethanol production.

### BRIEF DESCRIPTION OF THE FIGURES

FIG. 1 is a schematic of a two-stage pretreatment process. In the first stage, the feedstock is treated with, for example, a low severity steam treatment, autohydrolysis, or controlled pH pretreatment (Ladisch et al. U.S. Pat. No. 5,846,787). In the second stage, the substrate is treated with dilute acid at relatively low temperatures. Solids and/or hydrolyzate may then be recovered for further processing.

FIG. 2 shows glucose yields for enzymatic hydrolysis of MS028 and MS029 after subjecting the pretreated material to a second pretreatment step (dilute acid hydrolysis second pretreatment; 0.91% and 0.45% H₂SO₄). The controls were not subject to a dilute acid hydrolysis second pretreatment step before being subject to enzymatic hydrolysis.

FIG. 3 shows xylose yields for enzymatic hydrolysis of MS028 and MS029 after subjecting the pretreated material to a second pretreatment step (dilute acid hydrolysis second pretreatment; 0.91% H₂SO₄, 121° C., 60 min and 0.45% H₂SO₄, 121° C., 120 min). The controls were not subject to a dilute acid hydrolysis second pretreatment step before being subject to enzymatic hydrolysis. The white bars depict the xylose yield after the dilute acid hydrolysis second pretreatment step; the black bars depict the increase in the xylose yield upon subsequent enzymatic hydrolysis treatment of the pretreated material.

FIG. 4 shows glucose yields for enzymatic hydrolysis of MS028 and MS029 after subjecting the pretreated material to a second pretreatment step (dilute acid hydrolysis second pretreatment; 0.91% H₂SO₄, 121° C., 60 min and 0.45% H₂SO₄, 121° C., 120 min). The controls were not subject to a dilute acid hydrolysis second pretreatment step before being subject to enzymatic hydrolysis. The white bars depict the glucose yield after the dilute acid hydrolysis second pretreatment step; the black bars depict the increase in the glucose yield upon subsequent enzymatic hydrolysis treatment of the pretreated material.

FIG. 5 shows xylose yields for enzymatic hydrolysis of MS029 after subjecting the pretreated material to a second pretreatment step (dilute acid hydrolysis second pretreatment; 0.1%-0.4% H₂SO₄, 121° C., 2-10 h) at a relatively low solids concentration (9 wt %). The control was not subject to a dilute acid hydrolysis second pretreatment step before being subject to enzymatic hydrolysis. The white bars depict the xylose yield after the dilute acid hydrolysis second pretreatment step; the black bars depict the increase in the xylose yield upon subsequent enzymatic hydrolysis treatment of the pretreated material.

FIG. 6 shows glucose yields for enzymatic hydrolysis of MS029 after subjecting the pretreated material to a second pretreatment step (dilute acid hydrolysis second pretreatment; 0.1%-0.4% H₂SO₄, 121° C., 2-10 h) at a relatively low solids concentration (9 wt %). The control was not subject to a dilute acid hydrolysis second pretreatment step before being subject to enzymatic hydrolysis. The white bars depict the glucose yield after the dilute acid hydrolysis second pretreatment step; the black bars depict the increase in the glucose yield upon subsequent enzymatic hydrolysis treatment of the pretreated material.

FIG. 7 shows glucose yields for enzymatic hydrolysis of MS029 after subjecting the pretreated material to a second pretreatment step, at relatively high solids concentrations. The control was not subject to a dilute acid hydrolysis second pretreatment step before being subject to enzymatic hydrolysis.

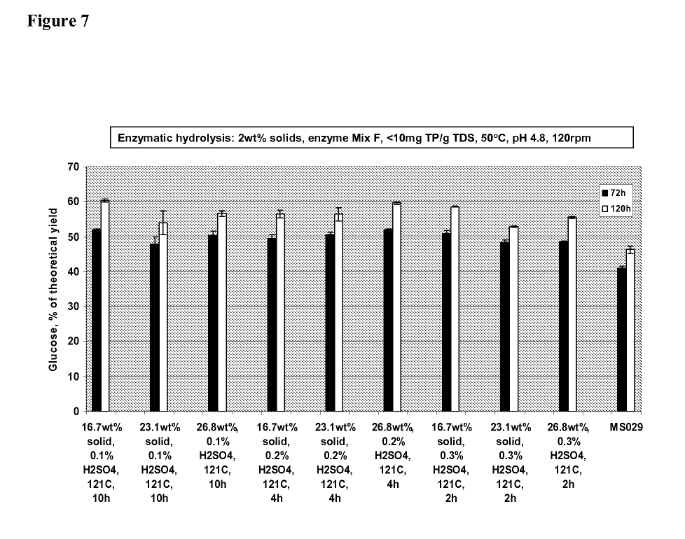

FIG. 8 shows xylose yields for enzymatic hydrolysis of MS029 after subjecting the pretreated material to a second pretreatment step (dilute acid hydrolysis second pretreatment; 0.1%-0.3% H₂SO₄, 121° C., 2-10 h) at a high solids concentration (16.7-26.8 wt %). The control was not subject to a dilute acid hydrolysis second pretreatment step before being subject to enzymatic hydrolysis. The white bars depict the yield of xylose monomer after the dilute acid hydrolysis second pretreatment step; the gray bars depict the yield of xylose oligomers after the dilute acid hydrolysis second pretreatment step; and the black bars depict the increase in the xylose yield upon subsequent enzymatic hydrolysis treatment of the pretreated material.

FIG. 9 shows glucose yields for enzymatic hydrolysis of MS029 after subjecting the pretreated material to a second pretreatment step (dilute acid hydrolysis second pretreatment; 0.1%-0.3% H₂SO₄, 121° C., 2-10 h) at a high solids concentration (16.7-26.8 wt %). The control was not subject to a dilute acid hydrolysis second pretreatment step before being subject to enzymatic hydrolysis. The white bars depict the yield of glucose monomer after the dilute acid hydrolysis second pretreatment step; the gray bars depict the yield of glucose oligomers after the dilute acid hydrolysis second pretreatment step; and the black bars depict the increase in the glucose yield upon subsequent enzymatic hydrolysis treatment of the pretreated material.

FIG. 10 summarizes the total xylose and total glucose yields (g), based on original total solids after subjecting the pretreated material to a second pretreatment step. The controls were not subject to a dilute acid hydrolysis second pretreatment step before being subject to enzymatic hydrolysis. The data show a significant increase in total glucose yield and a minimal increase in total xylose yield when the material is subject to a dilute acid hydrolysis second pretreatment step, compared to the controls.

FIG. 11 depicts the amount of sugar released when the second pretreatment step is a dilute acid hydrolysis second pretreatment step utilizing a low acid concentration (0.05% H₂SO₄) and very high temperatures (200° C.) (right), in comparison to when no second pretreatment step is used (left, control) and when the second pretreatment step is an autohydrolysis second pretreatment step (middle, hot water, 200° C., 12 min).

### DETAILED DESCRIPTION

### Overview

In certain embodiments, low-severity steam treatment is first applied to hemicellulosic biomass to break down gently hemicellulose and lignin, producing an intermediate substrate that is more accessible to acid for hemicellulose hydrolysis and lignin solubilization. In certain embodiments, autohydrolysis is first employed in order to gently break down the hemicellulose and lignin found in hemicellulosic biomass, producing an intermediate substrate that is more accessible to acid for hemicellulose hydrolysis and lignin solubilization. In some embodiments, the material may be further refined after low-severity steam treatment or autohydrolysis to reduce the particle size. In certain other embodiments, the material may be washed after low-severity steam treatment or autohydrolysis to reduce the concentrations of enzymatic inhibitors or inhibitors of microorganisms that may be solubilized or produced during the treatment.

In certain embodiments, complete hemicellulose hydrolysis may be carried out during the second stage of the pretreatment under mild conditions (e.g., dilute acid or hot water). Performing this step of the process under mild conditions may have the effect of reducing the degradation of hemicellulose sugars and the formation of inhibitors of enzymatic and microbial activity, each of which may be produced in problematic amounts when harsher pretreatment conditions are employed.

In some embodiments, the methods described herein lead to greater solubilization of lignin and generate highly digestible cellulose, which then requires a lower concentration of enzyme for processing. The solubilized lignin produced via the two-stage process described herein may be less degraded than the lignin produced via other, harsher, pretreatment methods. Moreover, the relatively mild processing conditions (low acid concentration, low temperature, low pressure) used in the invention may enable a practitioner to use relatively inexpensive material for reactor construction, as compared to the materials used to construct reactors suitable for harsher pretreatment methods.

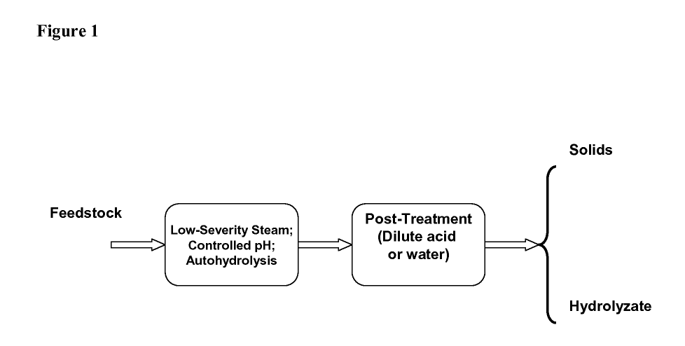

In certain embodiments, the two-stage pretreatment process of the present invention can be described schematically as shown in FIG. 1. In the process depicted in FIG. 1, lignocellulosic biomass may first be treated with a low-severity steam treatment to increase the porosity of the biomass structure and break down some fraction of the hemicellulose and lignin. The first step of the pretreatment may also be carried out via autohydrolysis or controlled pH pretreatment (see U.S. Pat. No. 5,846,787; incorporated by reference).

Low severity processes (for example, about 160 to 220° C. and severity ranging from 3.2 to 4.0) are used in the first stage of the pretreatment to prevent the loss of hemicellulose-derived sugars, as may occur during harsher treatments, such as steam explosion. Very dilute acids, very dilute bases, or other chemicals may be utilized during the first step of the pretreatment. In the second stage of the pretreatment method of the invention, dilute acid is added to the substrate recovered from the first stage. The dilute acid hydrolyzes hemicellulose and oligomeric sugars, while also solubilizing more lignin, further increasing the enzymatic digestibility of the cellulose. Low acid concentrations (e.g., about 0.02% to about 1 wt %) and mild temperatures (e.g., about 120° C. to about 220° C.) may be used in the second stage of the pretreatment process. Generally, hemicellulose becomes more susceptible to acid-mediated hydrolysis as its particle size and degree of polymerization decrease; in certain embodiments, these parameters may be varied to obtain efficient acid-mediated hydrolysis of a substrate. Dilute bases, organic solvents, or other chemicals may also be utilized during or after the second stage of the pretreatment methods. The second stage of the pretreatment may also be carried out solely in the presence of hot water.

After the first or second stage of the pretreatment process, solids and liquid may but need not be separated, depending on processing parameters (e.g., acid concentration) and subsequent treatment steps (e.g., enzymatic hydrolysis or fermentation). Due to the mild conditions used in the pretreatment steps, this process achieves higher hemicellulose sugar yields with less hemicellulose degradation, higher substrate digestibility with more lignin removal, and higher hydrolyzate fermentability with reduced formation and solubilization of inhibitors of enzymatic or microbial activity. In certain embodiments, a solid-liquid separation is carried out before the second stage of the pretreatment.

### Steam Pretreatment

Discontinuous steam explosion treatment was patented in 1929 by Mason (U.S. Pat. No. 1,655,618, hereby incorporated by reference in its entirety) for the production of boards of timber. The method combines a steam treatment with mechanical disorganization of lignocellulosic materials. In this process, wooden splinters are treated with steam at a pressure of 3.5 MPa or higher in a vertical steel cylinder. Once the treatment is completed, the material is discharged from the base of the cylinder. This harsh process combines the effects on the lignocellulosic material of high pressures and temperatures together with the final and sudden decompression. This treatment results in a combination of physical (segregation and rupture of the lignocellulosic materials) and chemical (de-polymerization and rupture of the C—O—C links) modifications. During steam treatment, most of the hemicellulose is hydrolyzed to water-soluble oligomers and free sugars.

Steam explosion treatment has a range of applications. For example, U.S. Pat. No. 4,136,207, hereby incorporated by reference in its entirety, describes the use of this kind of pretreatment to increase the digestibility of hard woods, such as poplar and birch, by ruminants. In this case, STAKE technology is used, operating continuously in a high-pressure tubular reactor at temperatures between 200° C. and 250° C. and for various treatment times. In the discontinuous steam explosion process developed by IOTECH Corporation, known alternatively as “flash hydrolysis” and the “IOTECH process”, the wood is ground to a small particle size and subjected to temperatures and pressures close to 230° C. and 500 psi; once these conditions are reached, it is suddenly discharged from the reactor. The wood's organic acids control the pH and acetic acid is present in the gaseous effluent. The design of the reactor is described in U.S. Pat. No. 4,461,648, hereby incorporated by reference. Additionally, Canadian patent CA 1,212,505 describes the application of a combination of the STAKE and IOTECH steam explosion processes to obtain paper paste from hard wood with high yields.

The fundamental objective of pretreatment is to reduce the crystallinity of the cellulose and to dissociate the hemicellulose-cellulose complex. The digestibility of the cellulose typically increases with the degree of severity of the pretreatment. This increase in digestibility is directly related to the increase in the available surface area (ASA) of the cellulose materials, which facilitates the eventual enzymatic attack by cellulases.

### Low-Severity (3.0-3.9) Steam Pretreatment

The increased accessibility of the substrate after steam pretreatment treatment appears to be due to changes in the distribution of pore size, the degree of crystallinity, the degree of polymerization and/or the residual xylan content, which determine its final effectiveness (K. K. Y. Wong et al., Biotechnol. Bioeng. 31, 447 (1988); H. L. Chum et al., Biotechnol. Bioeng. 31, 643, (1988)). While early researchers focused their work on the effects of sudden de-pressurization on the rupture of cellulose bonds in experiments at high temperatures (220° C. to 270° C.) and short treatment times (40 seconds to 90 seconds), more recent work (Wright, J. D. SERI/TP-231-3310, 1988; Schwald et al., in: Steam explosion Techniques. Fundamentals and Industrial Applications, Facher, Marzetti and Crecenzy (eds.), pages 308-320 (1989)), has shown that the use of relatively lower temperatures (no higher than 200° C. to 220° C.) and longer treatment times (5 minutes to 10 minutes) produces appropriate solubilization rates and also avoids the possibility of a certain amount of pyrolysis, which could give rise to inhibitory products. This milder approach leads to a greater recovery of glucose in the residue (Ballesteros et al., in: Biomass for Energy, Environment, Agriculture and Industry, Chartier, Beenackers and Grassi (eds.), Vol. 3., pages 1953-1958 (1995)). Further, an acidic catalyst may be added, to aid in the decomposition of lignocellulosic biomass. For example, sulfur dioxide may be used as a catalyst in steam pretreatment of lignocellulosic biomass. See, for example, Schell, D. J. et al. Applied Biochemistry and Biotechnology 28/29, 87-97 (1991).

### Controlled pH Pretreatment

A controlled pH pretreatment has been described by Ladisch et al. (U.S. Pat. No. 5,846,787, incorporated by reference in its entirety). This process involves the treatment of cellulosic materials with liquid water at a temperature greater than the glass transition temperature of the material, but not substantially exceeding 220° C., while maintaining the pH of the medium in a range that avoids substantial autohydrolysis of the cellulosic material. Such pretreatments minimize chemical changes to the cellulose while leading to physical changes which substantially increase the susceptibility to hydrolysis in the presence of cellulase. In certain embodiments, controlled pH pretreatment may be used as the first process of the two-stage pretreatment process described herein.

### Autohydrolysis

Autohydrolysis, also called compressed hot water pretreatment or steam pretreatment, is a process in which no chemicals are used. Acetic acid released during hemicellulose hydrolysis is often considered to be the catalyst for enhanced pretreatment. However, autohydrolysis suffers from slow reaction times because of the low concentration of acetic acid released. To increase the rate of autohydrolysis, high temperatures (200-230° C.) are generally required. However, high temperature operation will increase hemicellulose sugar degradation and lignin condensation which, in turn, will impact subsequent enzymatic hydrolysis processes. Additionally, total sugar recovery will be decreased (Heitz et al. 1991; Saddler et al. 1993). Flow-through pretreatment, on the other hand, uses just compressed hot water without elevated temperatures and can significantly increase hemicellulose sugar recovery and cellulose digestibility (Liu and Wyman 2005). However, flow-through pretreatment utilizes a large amount of water and has high energy requirements for both pretreatment and downstream processes, as the hemicellulose hydrolyzate is very dilute. Partial flow of compressed hot water through lignocellulosic biomass can combine some of the best features of flow-through and batch operations (Liu and Wyman 2003), but may still suffer from high operational costs.

### Lignocellulosic Material

The terms “lignocellulosic material” and “lignocellulosic substrate” mean any type of biomass comprising cellulose, such as but not limited to non-woody-plant biomass, agricultural wastes, forestry residues, paper-production sludge, waste-water-treatment sludge, and sugar-processing residues. Generally, a lignocellulosic material, on a dry basis, contains cellulose in an amount greater than about 25% (w/w), about 15% hemicellulose, and about 15% lignin. The lignocellulosic material can also be of higher cellulose content, for example, at least about 30% (w/w), 35% (w/w), 40% (w/w) or more.

In a non-limiting example, the lignocellulosic material can include, but is not limited to, grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, wheat straw, canola straw, oat straw, oat hulls, and corn fiber; stover, such as but not limited to soybean stover, corn stover; and forestry wastes, such as but not limited to recycled wood pulp fiber, sawdust, hardwood, softwood, or any combination thereof. Lignocellulosic material may comprise one species of fiber, or alternatively lignocellulosic material may comprise a mixture of fibers that originate from different lignocellulosic materials. In certain embodiments, lignocellulosic materials are agricultural wastes, such as cereal straws, including wheat straw, barley straw, canola straw and oat straw; stovers, such as corn stover and soybean stover; grasses, such as switch grass, reed canary grass, cord grass, and miscanthus; or combinations thereof.

The size range of the substrate material varies widely and depends upon the type of substrate material used as well as the requirements and needs of a given process. In one embodiment of the invention, the lignocellulosic raw material may be prepared in such a way as to permit ease of handling in conveyors, hoppers and the like. In the case of wood, the chips obtained from commercial chippers are suitable; in the case of straw it is sometimes desirable to chop the stalks into uniform pieces about 0.5-3 inches in length. Depending on the intended degree of pretreatment, the size of the substrate particles prior to pretreatment may range from less than a millimeter to inches in length. The particles need only be of a size that is reactive.

### Reactors and Reaction Conditions

The terms “reactor” and “pretreatment reactor” mean any vessel suitable for practicing a method of the present invention. The dimensions of the pretreatment reactor should be sufficient to accommodate the lignocellulose material conveyed into and out of the reactor, as well as additional headspace around the material. In a non-limiting example, the headspace extends about one foot to about four feet around the space occupied by the materials. Furthermore, the pretreatment reactor should be constructed of a material capable of withstanding the pretreatment conditions. Specifically, the construction of the reactor should be such that the pH, temperature and pressure do not affect the integrity of the vessel. For example, the reactor may be run at temperatures corresponding to saturated steam pressures of about 10 psig to about 400 psig, and in the presence of an acid, for example, sulfuric acid (see U.S. Pat. No. 4,461,648, which is incorporated herein by reference in its entirety).

In a non-limiting example of the present invention, the lignocellulosic materials may be soaked in water or other suitable liquid(s) prior to the addition of steam or acid or both. The excess water may be drained from the lignocellulosic materials. The soaking may be performed prior to conveying into the reactor, or subsequent to entry (i.e., inside the pretreatment reactor). Without wishing to be bound by theory, soaking the materials may help promote better penetration of the steam during the first stage of the pretreatment process.

In certain embodiments, steam is added to the reactor at a saturated steam pressure of between about 10 psig and about 400 psig, or any amount there between; for example, the saturated steam pressure may be about 10, 20, 30, 45, 60, 75, 100, 150, 200, 250, 300, 350, or 400 psig.

In the second stage of the pretreatment process, the biomass may be treated with acid. The acid used in the method of the present invention may be any suitable acid known in the art; for example, but without wishing to be limiting in any manner, the acid may be sulfuric acid, sulfurous acid, sulfur dioxide, H₃PO₄, H₂CO₃, or a combination thereof. The amount of acid added may be any amount sufficient to provide a good pretreatment of the lignocellulosic material at the chosen pretreatment temperature. For example, but without wishing to be limiting, the acid loading may be about 0% to about 1% by weight of the materials, or any amount there between; for example, the acid may be loaded at about 0, 0.02, 0.04, 0.05, 0.06, 0.08, 0.1, 0.2, 0.3, 0.4, 0.5, 0.6, 0.7, 0.8, 0.9, 1.0, 1.1, or 1.2% by weight of the lignocellulosic materials, depending on the feedstock. In a non-limiting example, the acid is sulfur dioxide, and it is added to the lignocellulosic material by injecting the acid as a vapor to a concentration of about 0.02% to about 1.0% the weight of lignocellulosic material.

In the second stage of the pretreatment process, the biomass may be treated with hot water. The temperature of the water in this step may range from about 80° C. to about 220 ° C., or from about 100° C. to about 130° C., or from about 115° C. to about 130° C., or from about 180° C. to 220° C.

During each stage of the pretreatment process, the reactor may be maintained at a specific temperature and pH for a length of time sufficient to hydrolyze a portion of the hemicellulose. The combination of time, temperature, and pH may be any suitable conditions known in the art. In a non-limiting example, the temperature, time and pH may be as described in U.S. Pat. No. 4,461,648, which is hereby incorporated by reference.

The temperature may be about 115° C. to about 230° C., or any temperature there between. More specifically, the temperature may be about 115° C. to about 130° C., or about 130° C. to about 190° C., or about 180° C. to about 220° C., or any temperature therebetween. For example, the temperature may be about 115, 120, 121, 125, 130, 135, 140, 145, 150, 155, 160, 165, 170, 175, 180, 185, 190, 195, 200, 205, 210, 215, or 220° C. Those skilled in the art will recognize that the temperature can vary within this range during the pretreatment. The temperatures refer to the approximate temperature of the process material reactor, recognizing that at a particular location the temperature may be higher or lower than the average temperature.

The pH in the pretreatment reactor may be maintained from about 1.5 to about 6.0, or any pH therebetween; for example, the pH may be about 1.5, 1.8, 2.0, 2.2, 2.5, 3.0, 3.5, 4.0, 4.5, 5.0, 5.5, or 6.0. In a non-limiting example, the pH in the pretreatment reactor is about 1.5 to about 2.5, or about 2.5 to about 4.0. To achieve a pH within the specified range, generally about 0% to about 1% weight of acid on weight of solids must be added to the lignocellulose materials.

The concentration of solids used in the pretreatment stages may be maintained from about 2 wt % to about 30 wt %. In certain embodiments, the concentration of solids used in any of the pretreatment stages may be about 2, 4, 6, 8, 10, 12, 14, 16, 18, 20, 22, 24, 26, 28, or 30 wt %. In other embodiments, the concentration of solids used in any of the pretreatment stages may be about 9, 16.7, 23.1, or 26.8 wt %.

While the methods described above, in some instances, pertain to a batch reactor assembly, the inventive methods should be in no way limited to such an assembly. In addition, a combination of batch and continuous processes may be used.

In certain embodiments, the present invention relates to the aforementioned method, wherein said lignocellulosic material, on a dry basis, contains at least about 25% (w/w) cellulose, at least about 15% (w/w) hemicellulose, and at least about 15% (w/w) lignin.

In certain embodiments, the present invention relates to the aforementioned method, wherein said lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood, and combinations thereof.

In certain embodiments, the present invention relates to the aforementioned method, wherein there is only one pretreatment reactor.

In certain embodiments, the present invention relates to the aforementioned method, further comprising the step or steps of transferring the material through one or more additional reactors.

In certain embodiments, the present invention relates to the aforementioned method, wherein the first pretreatment step is conducted in a first reactor; and the second pretreatment step is conducted in a second reactor.

In certain embodiments, the present invention relates to the aforementioned method, wherein said lignocellulosic material contains, on a dry basis, at least about 25% (w/w) cellulose, at least about 15% (w/w) hemicellulose, and at least about 15% (w/w) lignin.

In certain embodiments, the present invention relates to the aforementioned method, wherein said lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood, and combinations thereof.

In certain embodiments, the present invention relates to the aforementioned method, wherein said lignocellulosic material is heated prior to pretreatment.

In certain embodiments, the present invention relates to the aforementioned method, wherein said reactor is sealed before said injection of steam or acid.

In certain embodiments, the present invention relates to the aforementioned method, wherein air is removed from said reactor, thereby creating a vacuum.

### METHODS OF THE INVENTION

In certain embodiments, the invention relates to a method for pre-treating lignocellulosic material, comprising:

exposing the lignocellulosic material to a low-severity first pretreatment step to give a first product; and

contacting said first product with dilute aqueous acid or hot water to give a second product.

In certain embodiments, the invention relates to any one of the aforementioned methods, wherein the low-severity first pretreatment is at a temperature from about 160° C. to about 220° C.

In certain embodiments, the invention relates to any one of the aforementioned methods, wherein the severity of the low-severity first pretreatment step is about 3.2 to about 4.0.

In certain embodiments, the invention relates to any one of the aforementioned methods, wherein the low-severity first pretreatment is at a temperature from about 160° C. to about 220° C. and the severity is about 3.2 to about 4.0.

In certain embodiments, the invention relates to any one of the aforementioned methods, wherein the first product is contacted with hot water at a temperature from about 100° C. to about 140° C.

In certain embodiments, the invention relates to any one of the aforementioned methods, wherein the first product is contacted with hot water at a temperature from about 180° C. to about 220° C.

In certain embodiments, the invention relates to any one of the aforementioned methods, wherein the low-severity first pretreatment is at a temperature from about 160° C. to about 220° C. and the severity is about 3.2 to about 4.0; and the first product is contacted with hot water at a temperature from about 100° C. to about 140° C.

In certain embodiments, the invention relates to a method for pre-treating lignocellulosic material, comprising:

exposing the lignocellulosic material to a low-severity first pretreatment step to give a first product; and

contacting said first product with dilute aqueous acid to give a second product.

In certain embodiments, the invention relates to any one of the aforementioned methods, wherein the low-severity first pretreatment step is selected from the group consisting of steam treatment, autohydrolysis, and controlled pH pretreatment.

In certain embodiments, the invention relates to the aforementioned method, wherein the dilute aqueous acid is selected from the group consisting of sulfuric acid, sulfurous acid, sulfur dioxide, H₃PO₄, and H₂CO₃. In certain embodiments, the invention relates to the aforementioned method, wherein the low severity pretreatment step is steam treatment, and the conditions under which the steam treatment occurs are: from about 160° C. to about 230° C., from about 75 psig to about 400 psig, and from about 1 min to about 60 min.

In certain embodiments, the invention relates to the aforementioned method, wherein the low severity pretreatment step is controlled pH pretreatment; and the controlled pH pretreatment step comprises heating in liquid water the lignocellulosic material at or above its glass transition temperature, while not exceeding 220° C., while maintaining the pH of the medium in a range that avoids substantial autohydrolysis of the cellulosic material.

In certain embodiments, the invention relates to any one of the aforementioned methods, wherein the susceptibility to hydrolysis by an enzyme of the cellulose within the second product is greater than that of cellulose in the lignocellulosic material.

In certain embodiments, the invention relates to any one of the aforementioned methods, further comprising the step of exposing the second product to an enzyme. In certain embodiments, the invention relates to the aforementioned method, wherein the enzyme comprises cellulase, beta-glucosidase, or xylanase.

In certain embodiments, the invention relates to any one of the aforementioned methods, wherein the lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood, and combinations thereof.

In certain embodiments, the invention relates to any one of the aforementioned methods, wherein said lignocellulosic material contains, on a dry basis, at least about 25% (w/w) cellulose, at least about 15% (w/w) hemicellulose, and at least about 15% (w/w) lignin.

In certain embodiments, the invention relates to any one of the aforementioned methods, wherein the method is conducted in one pretreatment reactor.

In certain embodiments, the invention relates to any one of the aforementioned methods, further comprising the step of transferring the lignocellulosic material, the first product, or the second product through a plurality of reactors. In certain embodiments, the invention relates to any one of the aforementioned methods, wherein the first pretreatment step is conducted in a first reactor; and the second pretreatment step is conducted in a second reactor.

In certain embodiments, the invention relates to any one of the aforementioned methods, wherein the concentration of the acid is about 0.02 wt % to about 1 wt %. In certain embodiments, the invention relates to any one of the aforementioned methods, wherein the concentration of the acid is about 0.05 wt % to about 0.91 wt %. In certain embodiments, the invention relates to any one of the aforementioned methods, wherein the concentration of the acid is about 0.05 wt % to about 0.45 wt %. In certain embodiments, the invention relates to any one of the aforementioned methods, wherein the concentration of the acid is about 0.05 wt % to about 0.4 wt %. In certain embodiments, the invention relates to any one of the aforementioned methods, wherein the concentration of the acid is about 0.05 wt % to about 0.3 wt %. In certain embodiments, the invention relates to any one of the aforementioned methods, wherein the concentration of the acid is about 0.05 wt % to about 0.2 wt %. In certain embodiments, the invention relates to any one of the aforementioned methods, wherein the concentration of the acid is about 0.1 wt %. In certain embodiments, the invention relates to any one of the aforementioned methods, wherein the concentration of the acid is about 0.05 wt %.

In certain embodiments, the invention relates to any one of the aforementioned methods, wherein the treatment with the dilute acid is performed for about 0.1 hour to about 10 hours. In certain embodiments, the invention relates to any one of the aforementioned methods, wherein the treatment with the dilute acid is performed for about 1 hour to about 10 hours. In certain embodiments, the invention relates to any one of the aforementioned methods, wherein the treatment with the dilute acid is performed for about 1 hour to about 4 hours. In certain embodiments, the invention relates to any one of the aforementioned methods, wherein the treatment with the dilute acid is performed for about 1 hour to about 2 hours. In certain embodiments, the invention relates to any one of the aforementioned methods, wherein the treatment with the dilute acid is performed for about 0.1 hour to about 0.5 hours. In certain embodiments, the invention relates to any one of the aforementioned methods, wherein the treatment with the dilute acid is performed for 0.2 h.

In certain embodiments, the invention relates to any one of the aforementioned methods, wherein the solids concentration prior to pretreatment is about 9 wt % to about 26.8 wt %. In certain embodiments, the invention relates to any one of the aforementioned methods, wherein the solids concentration prior to pretreatment is about 9 wt % to about 23.1 wt %. In certain embodiments, the invention relates to any one of the aforementioned methods, wherein the solids concentration prior to pretreatment is about 9 wt % to about 16.7 wt %.

In certain embodiments, the invention relates to any one of the aforementioned methods, further comprising the step of separating the first product into a first liquid fraction and a first solid fraction.

In certain embodiments, the invention relates to any one of the aforementioned methods, further comprising the step of separating the second product into a second liquid fraction and a second solid fraction.

In certain embodiments, the invention relates to any one of the aforementioned methods, wherein said low-severity first pretreatment step comprises dilute acid or dilute base.

In certain embodiments, the invention relates to any one of the aforementioned methods, wherein said low-severity first pretreatment step comprises dilute base.

### EXEMPLIFICATION

### Enhancing Enzymatic Hydrolysis of Steamed Hardwood by Subsequent Mild Treatment

Steam explosion and autohydrolysis each have the ability rapidly to reduce particle sizes, open biomass structure, and degrade hemicellulose and lignin in hemicellulosic biomass. However, depending on the severity of the treatment, depolymerization, degradation, and decrystallization of the cellulose may also occur. Moreover, although some soluble sugar monomers and low degree of polymerization (DP) oligomers are produced, the majority of the hemicellulose and lignin output from these treatments exists as high-DP sugar oligomers or high molecular weight (MW) lignin-carbohydrate compounds (LCC). The high-DP oligomeric sugars and high MW LCC are less soluble or insoluble, and can prevent approach of enzymes to cellulose, reducing sugar yields. In addition, a previous study showed that these compounds may be the key inhibitors to enzymes.

### Materials & Methods

Substrates. MS028 and MS029 are hardwood pretreated by steam explosion at different severities. Both substrates were unwashed, mixed hardwood substrates from steam explosion or autohydrolysis at a relatively low severity of about 3.29 and about 3.59, respectively. The moisture content of both substrates was about 50%.

Enzymes. “Enzyme Mix F” is an enzyme cocktail made of spezyme cellulase

(GENENCOR), xylanase (MULTIFACT), and beta-glucosidase (NOVOZYME 188) at a protein ratio of 5:1:1.

“Enzyme Mix B” is an enzyme cocktail made of AB enzyme monocomponents (CBH1, EG, xylanase, and beta-glucosidase) at a protein ratio of 5:1.54:0.14:0.16.

Dilute Acid Treatment. MS028 or MS029 were loaded in a reagent bottle and mixed with H₂SO₄ or DI water at different solids concentrations. The bottle was then autoclaved at 121° C., for various times. After autoclaving, solid and liquid hydrolyzate were separated by filtration and hot washing (50° C.-60° C. DI water). The liquid fraction was stored at 4° C. for sugar analysis. The solids were frozen and used as substrate for enzymatic hydrolysis.

Enzymatic Hydrolysis. Enzymatic hydrolysis was carried out in 120 mL flasks at various total solids concentrations. The enzyme dose was 10 mg total protein (TP) per gram total dry solid (TDS), or 10 mg TP/g TDS. Enzymatic hydrolysis conditions were: 2 wt % solids, 50° C., ph 4.8, 72 h, 120 rpm.

Sugar Analysis. Monomeric sugars and cellobiose were analyzed by HPLC, using a Bio-Rad Amine HPX-87P column. Total xylose (both monomeric xylose and xylo-oligomers in the liquid fraction were quantified after subsequent treatment with H₂SO₄ (4 wt % at 121° C., for 1 h)).

Treatment with 0.45-0.91% H₂SO₄ at a Low Solid Concentration (9 wt % solid)

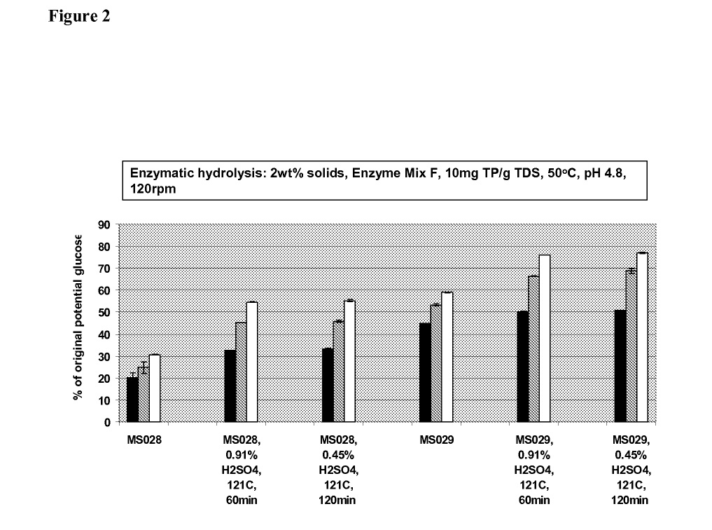

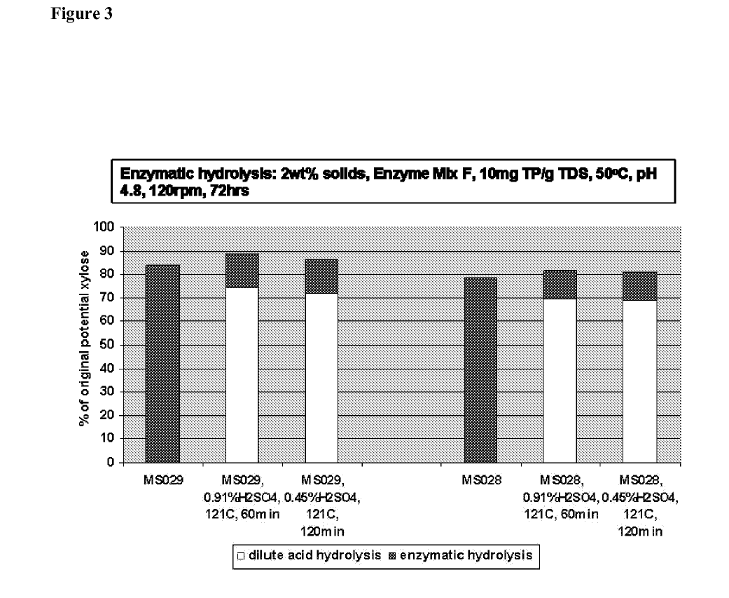

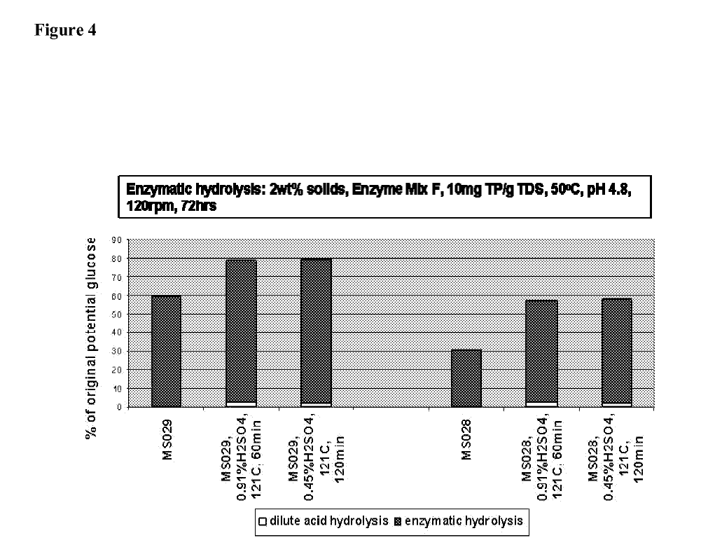

To evaluate the effects of a two-stage pretreatment process, MS028 and MS029 were subsequently treated with dilute acid. As shown in FIGS. 2-4, treatment of MS028 and MS029 with dilute acid significantly increased the total hemicellulose sugar yield and cellulose digestibility at the same enzyme dose. As presented in FIG. 4, when MS028 and MS029 were treated with 0.45-0.91 wt % sulfuric acid at 121° C., total glucose yields increased by about 20% (of theoretical yield) compared to the control (without subsequent treatment). However, total xylose yield did not change significantly (FIG. 3). Total glucose yield or total xylose yield was computed as follows: the yield of total glucose or xylose from both the subsequent acid treatment step and the enzymatic hydrolysis step.

Treatment With 0.1-0.4% H₂SO₄ at a Low Solid Concentration (9 wt % solid)

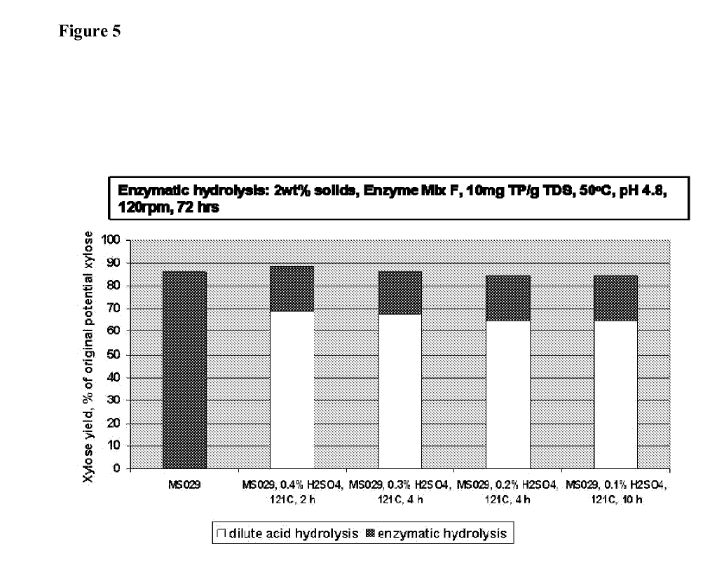

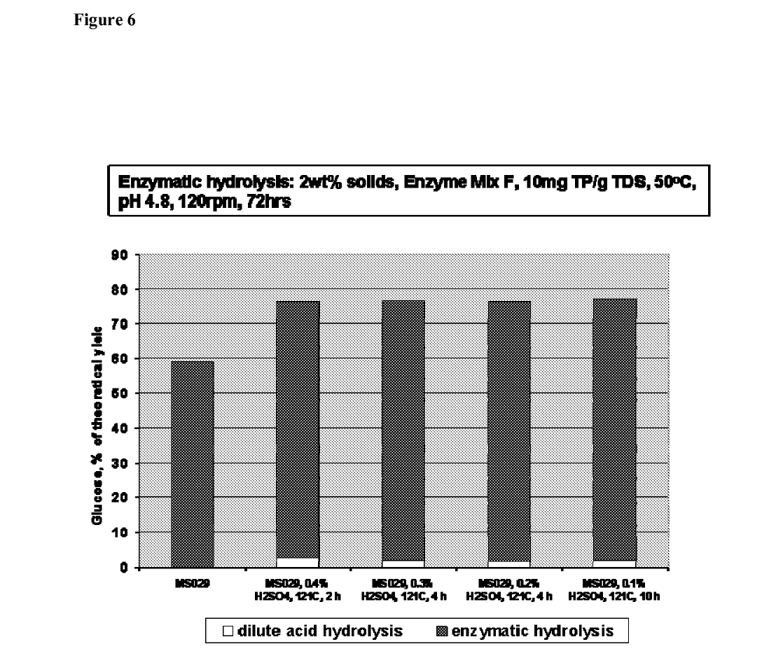

To further evaluate the effects of acid concentration and treatment time on performance of the second stage of treatment, MS029 at a relatively low solid concentration (9 wt %) was treated with 0.1-0.4% H₂SO₄, at 121° C. for various residence times. As illustrated by FIG. 6, subsequent treatment at such a low acid concentration can also significantly increase substrate digestibility. When MS029 was treated with 0.1-0.4% H₂SO₄ at 121° C. for 2 to 4 h, total glucose yield increased by about 15% (of theoretical yield), compared to the control. Again, subsequent treatment at these low acid concentrations did not significantly affect total xylose yield, as shown in FIG. 5.

### Treatment With 0.1-0.3% H₂50₄ at High Solid Concentration (16.7-26.8 wt %)

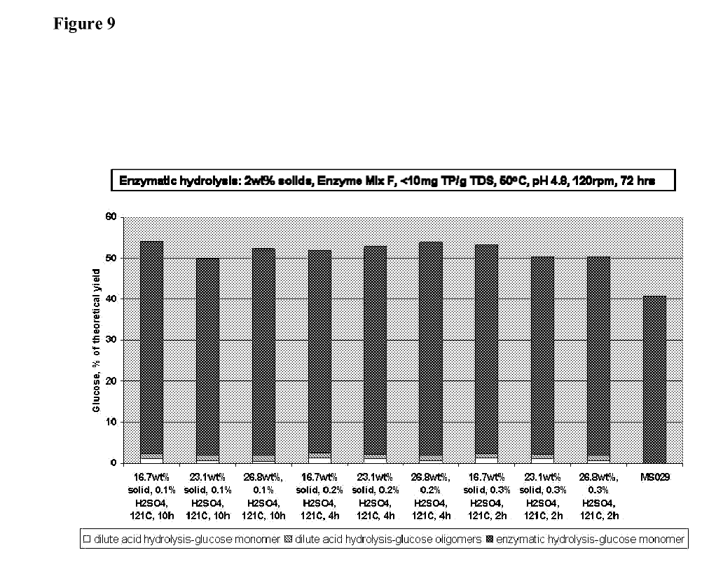

To evaluate the effect of solid concentration on the efficiency of subsequent acid treatment, MS029 at solid concentrations of 16.7 wt %, 23.1 wt %, or 26.8 wt % was treated with dilute acid (0.1-0.3 wt % H₂SO₄) at 121° C. for various residence times (2 to 10 h). As presented in FIG. 9, subsequent treatment of high solids samples also increased cellulose digestibility by approximately 10% (of the theoretical) for all cases, compared to the control (without post-treatment). In addition, total glucose yields did not change significantly for post-treatment at the same conditions (the same acid concentration and residence time) at higher solid concentrations, indicating that solid concentrations did not have a significant impact on performance of post-treatment in increasing substrate digestibility. It appeared that some xylose was lost in the second stage of treatment at high solid concentrations

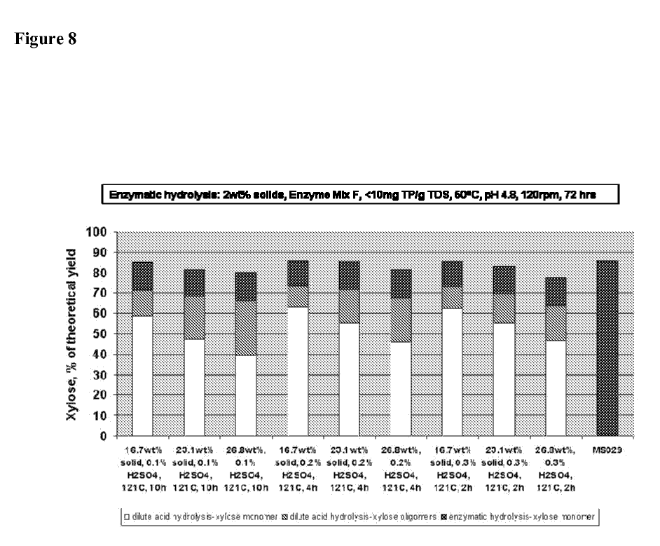

(FIG. 8). Xylose loss also was increased with increasing solid concentrations (FIG. 8). In addition, a large fraction of solubilized hemicellulose sugars was found to consist of xylose oligomers, which increased with increasing solids concentration in the second stage of treatment. For example, the subsequent treatment of 26.8 wt % solids with 0.1% H₂SO₄ resulted in approximately 45% of total xylose existing as xylose oligomers.

Further Analysis of Acid Pre-Treatment

The effects of dilute sulfuric acid (0.91% and 0.45% H₂SO₄) on hemicellulose hydrolysis and enzymatic digestibility of MS028 and MS029 were investigated further. The enzymatic hydrolysis conditions were 2% initial TS, 10 mg EP/g TS, 50° C.

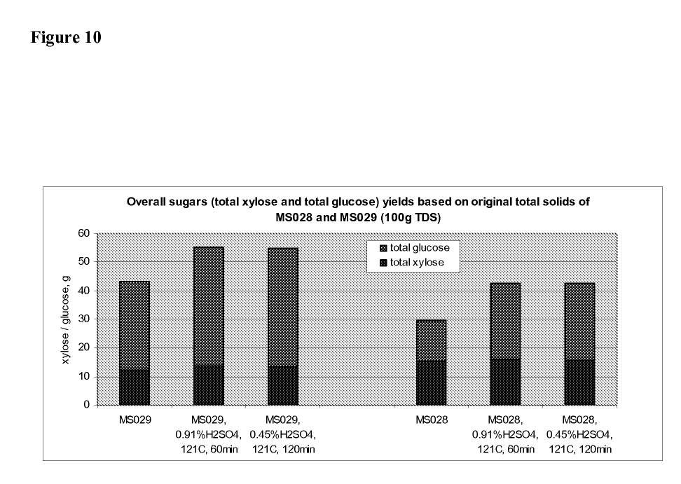

FIG. 10 summarizes the total xylose and total glucose yields, from MS029 and MS028, based on the original total solids concentration. The data further indicated that dilute treatment can significantly increase overall sugar (total xylose and total glucose) yields for both MS029 and MS028. Dilute acid treatment increased overall sugar yields from 43 to 55 g sugar/100 g substrate for MS029 and from 30 to 43 g sugar/100 g substrate for MS028. Based on these results, 550 kg sugars could be produced from 1 ton of MS029 (dry weight) by enzymatic hydrolysis at an enzyme dose of 10 TEP/g TDS, equivalent to a maximum yield of 93.8 gallon ethanol/ton TDS MS029.

### Treatment With 0.05% H₂SO₄

Pretreated mixed hardwood substrate (MS623) was washed at a ratio of liquid to solids of 20:1 to remove the soluble hemicellulose fraction. The solids were pretreated again using a Parr reactor at the conditions of 10 wt % solids, water or 0.05 wt % H₂SO₄, 200 ° C., and varying residence times (8-16 minutes). The whole slurry was then neutralized to pH 5.0, followed by composition analysis and digestibility tests.

Digestibility of the whole pretreated slurry was evaluated by enzymatic hydrolysis using Novozymes cellulase enzyme (Zoomerase, NS22c). The hydrolysis conditions were the same in each hydrolysis: 5 wt % total solids (TS), 5 mg total protein (TP) per gram total solids, pH 4.8, 35° C., and 72 h.

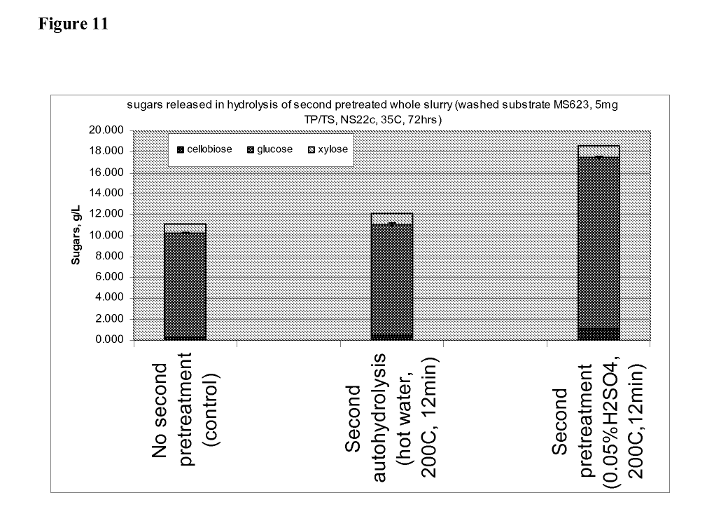

Second pretreatment with hot water or autohydrolysis can increase total sugar yield in enzymatic hydrolysis by ˜20%, compared to no second pretreatment. An important finding is that the addition of 0.05% H₂SO₄ in the second pretreatment tremendously improves substrate enzymatic digestibility. As presented in FIG. 11, total sugar release in enzymatic hydrolysis of 0.05% H₂SO₄-catalyzed second pretreated substrate increased by 80%, compared to the control substrate. It is possible that substrate digestibility can be further increased by optimizing the second pretreatment conditions.

### INCORPORATION BY REFERENCE

All of the U.S. patents and U.S. published patent applications cited herein are hereby incorporated by reference. In addition, U.S. Pat. No. 4,600,590 is hereby incorporated by reference; U.S. Pat. No. 5,037,663 is hereby incorporated by reference; U.S. Pat. No. 5,171,592 is hereby incorporated by reference; U.S. Pat. No. 5,473,061 is hereby incorporated by reference; U.S. Pat. No. 5,865,898 is hereby incorporated by reference; U.S. Pat. No. 5,939,544 is hereby incorporated by reference; U.S. Pat. No. 6,106,888 is hereby incorporated by reference; U.S. Pat. No. 6,176,176 is hereby incorporated by reference; U.S. Pat. No. 6,348,590 is hereby incorporated by reference; U.S. Pat. No. 6,392,035 is hereby incorporated by reference; U.S. Pat. No. 6,416,621 is hereby incorporated by reference; U.S. published patent application 2005/0065336 is hereby incorporated by reference; and U.S. published patent application 2006/0024801 is hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for pre-treating lignocellulosic material, comprising:
exposing the lignocellulosic material to a low-severity first pretreatment step to give a first product; and
contacting said first product with dilute aqueous acid to give a second product.

2. The method of claim 1, wherein the low-severity first pretreatment step is selected from the group consisting of steam treatment, autohydrolysis, and controlled pH pretreatment.

3. The method of claim 1, wherein the dilute aqueous acid is selected from the group consisting of sulfuric acid, sulfurous acid, sulfur dioxide, H₃PO₄, and H₂CO₃.

4. The method of claim 2, wherein the low severity pretreatment step is steam treatment, and the conditions under which the steam treatment occurs are: from about 160° C. to about 230° C., from about 75 psig to about 400 psig, and from about 1 min to about 60 min.

5. The method claim 2, wherein the low severity pretreatment step is controlled pH pretreatment; and the controlled pH pretreatment step comprises heating in liquid water the lignocellulosic material at or above its glass transition temperature, while not exceeding 220° C., while maintaining the pH of the medium in a range that avoids substantial autohydrolysis of the cellulosic material.

6. The method of claim 1, wherein the susceptibility to hydrolysis by an enzyme of the cellulose within the second product is greater than that of cellulose in the lignocellulosic material.

7. The method of claim 1, further comprising the step of exposing the second product to an enzyme.

8. The method of claim 7, wherein the enzyme comprises cellulase, beta-glucosidase, or xylanase.

9. The method of claim 1, wherein the lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood, and combinations thereof.

10. The method of claim 1, wherein said lignocellulosic material contains, on a dry basis, at least about 25% (w/w) cellulose, at least about 15% (w/w) hemicellulose, and at least about 15% (w/w) lignin.

11. The method of claim 1, wherein the method is conducted in one pretreatment reactor.

12. The method of claim 1, further comprising the step of transferring the lignocellulosic material, the first product, or the second product through a plurality of reactors.

13. The method of claim 1, wherein the first pretreatment step is conducted in a first reactor; and the second pretreatment step is conducted in a second reactor.

14. The method of claim 1, wherein the concentration of the acid is about 0.05 wt % to about 1 wt %.

15-20. (canceled)

21. The method of claim 1, wherein the treatment with the dilute acid is performed for about 1 hour to about 10 hours.

22-23. (canceled)

24. The method of claim 1, wherein the solids concentration prior to pretreatment is about 9 wt % to about 26.8 wt %.

25-26. (canceled)

27. The method of claim 1, further comprising the step of separating the first product into a first liquid fraction and a first solid fraction.

28. The method of claim 1, further comprising the step of separating the second product into a second liquid fraction and a second solid fraction.
